(19) United States
(12) Patent Application Publication
(10) Pub. No.: US 2025/0292890 A1
(43) Pub. Date: Sep. 18, 2025

(54) IMAGE PROCESSING APPARATUS, RADIATION IMAGING SYSTEM, IMAGE PROCESSING METHOD, AND COMPUTER-READABLE STORAGE MEDIUM

(71) Applicant: CANON KABUSHIKI KAISHA, Tokyo (JP)

(72) Inventor: Yuma HOSOYA, Tokyo (JP)

(21) Appl. No.: 19/221,677

(22) Filed: May 29, 2025

Related U.S. Application Data
(63) Continuation of application No. PCT/JP2023/042206, filed on Nov. 24, 2023.

(30) Foreign Application Priority Data
Nov. 30, 2022 (JP) 2022-191028

(51) Int. Cl. G16H 30/40; G06T 3/40; G06T 3/60; G06T 7/73; G16H 30/20

(52) U.S. Cl. CPC G16H 30/40; G06T 3/40; G06T 3/60; G06T 7/73; G16H 30/20

(57) ABSTRACT

An information processing apparatus is provided that comprises: an obtaining unit configured to obtain an optical image which is obtained by optically imaging a subject and a radiation image which is obtained by imaging the subject with radiation; and an image processing unit configured to perform image processing including at least one of rotation processing, extraction processing and scaling processing on the optical image so that the optical image and the radiation image correspond to each other.

### CROSS-REFERENCE TO RELATED APPLICATIONS

This application is a Continuation of International Patent Application No. PCT/JP2023/042206, filed Nov. 24, 2023, which claims the benefit of Japanese Patent Application No. 2022-191028, filed Nov. 30, 2022, both of which are hereby incorporated by reference herein in their entirety.

### BACKGROUND

### Field of the Technology

This disclosure relates to an image processing apparatus, a radiation imaging system, an image processing method, and a computer-readable storage medium.

### Description of the Related Art

In the medical field, a radiation imaging system using radiation is well known. Recently, due to the digitalization of the radiation imaging system, a system in which a radiation generating apparatus irradiates the radiation and a radiation imaging apparatus detects the entered radiation via a subject, and a digital radiation image is generated and displayed based on the detected radiation has become popular. In such a system, the user can confirm the image immediately after the radiation imaging by checking the displayed radiation image. Therefore, by using a digitized radiation imaging system, the workflow is improved compared with the conventional imaging method using a film, and the imaging can be performed at a faster cycle.

In the imaging with such a radiation imaging system, it is necessary to perform the positioning of a patient and the radiation imaging apparatus, which include an instruction of the posture of the patient, according to imaging-condition (an imaged site, a distance between an X-ray tube and a detector, etc.) set in the radiation imaging system in advance. Specifically, the patient is moved between the radiation generating apparatus and the radiation imaging apparatus, and the positioning is performed so that the imaged site of the patient is included in a region where the radiation is irradiated (irradiation field). If the positioning is completed, the user such as a physician and a radiation engineer temporarily leave the patient and operate the X-ray exposure in the operation room.

However, when the user performs the operation in the operation room, since the user is away from the patient, there is a problem that it is not easy to confirm whether the positioning is maintained. In this regard, PTL 1 discloses a technology in which a television camera is attached to a gantry of an X-ray CT apparatus, and a camera image captured by the camera is displayed on a monitor when the imaging is performed by the X-ray CT apparatus.

In general, the orientation of a radiation image output by the radiation imaging apparatus is set to be constant, and users such as a physician and a radiation engineer position the patient in consideration of the orientation of the output radiation image. However, since the appearances of the optical image obtained by the optical camera changes depending on the orientation and position of the patient, the image may be difficult for the user to check. In such cases, it is necessary to ask the patient to move or adjust the image.

### CITATION LIST

### Patent Literature

PTL 1 Japanese Patent Laid-Open No. 2009-119281

### SUMMARY

Therefore, in an aspect of the present disclosure, one of the purposes is to perform image processing on at least one of an optical image and a radiation image so that the optical image and the radiation image correspond to each other.

An image processing apparatus according to an aspect of the present disclosure comprises an obtaining unit configured to obtain an optical image which is obtained by optically imaging a subject and a radiation image which is obtained by imaging the subject with radiation; and an image processing unit configured to perform image processing including at least one of rotation processing, extraction processing and scaling processing on the optical image so that the optical image and the radiation image correspond to each other.

Features of the present disclosure will become apparent from the following description of exemplary embodiments with reference to the attached drawings. The following description of embodiments are described by way of example.

### BRIEF DESCRIPTION OF THE DRAWINGS

FIG. 1 is a diagram for illustrating an example of the schematic configuration of a radiation imaging system according to a first embodiment.

FIG. 2 is a diagram for illustrating an example of a setting screen of an imaging-protocol according to the first embodiment.

FIG. 3 is a flowchart showing image processing according to the first embodiment.

FIG. 4 is a diagram for explaining the image processing according to the first embodiment.

FIG. 5 is a flowchart showing imaging processing according to the first embodiment.

FIG. 6 is a diagram for illustrating an example of the schematic configuration of a radiation imaging system according to a second embodiment.

FIG. 7 is a flowchart showing imaging processing according to the second embodiment.

FIG. 8 is a diagram for illustrating an example of the schematic configuration of a radiation imaging system according to a third embodiment.

FIG. 9 is a diagram for illustrating an example of an imaging-protocol setting screen of an optical image according to the third embodiment.

FIG. 10 is a diagram for illustrating an example of an imaging-protocol setting screen of a radiation image according to the third embodiment.

FIG. 11 is a flowchart showing imaging preparation processing of the optical image according to the third embodiment.

FIG. 12 is a flowchart showing imaging preparation processing of the radiation image according to the third embodiment.

FIG. 13 is a flowchart showing imaging processing according to the third embodiment.

FIG. 14 is a diagram for illustrating an example of a display screen according to a fourth embodiment.

FIG. 15 is a flowchart showing imaging processing according to the fourth embodiment.

### DESCRIPTION OF THE EMBODIMENTS

Hereinafter, exemplary embodiments for implementing the present disclosure will now be described in detail with reference to the drawings. However, the dimensions, materials, shapes, relative positions, the like of the components described in the following embodiment and examples can be freely set and may be modified according to the configuration of the apparatus to which the present invention is applied or various conditions. Also, in the drawings, the same reference numerals are used between the drawings to indicate identical or functionally similar elements.

In the following, the term “radiation” may include, for example, electromagnetic radiation such as X-rays and γ-rays, and particle radiation such as α-rays, β-rays, particle rays, proton rays, heavy ion rays, and meson rays.

The term “machine learning model” refers to a learning model based on a machine learning algorithm. Specific algorithms of the machine learning include the nearest neighbor method, the naive Bayes method, the decision tree, the support vector machine, and the like. The deep learning, which generates characteristic amount and combining weighting factors for learning by itself using neural networks, is also mentioned. As an algorithm using the decision tree, a method using gradient boosting such as LightGBM or XGBoost is also mentioned. It can be applied to the following embodiments and modifications as appropriate using available algorithms among the above. The term “teacher data” refers to training data and consists of a pair of input data and output data. The output data of training data is also called ground truth.

Further, the term “learned model” refers to a machine learning model that has been performed training (learning) on a machine learning model, which accords to any machine learning algorithm such as the deep learning, using an appropriate training data (training data) in advance. The learned model has been obtained using an appropriate training data in advance, however it does not mean that further learning is not performed, and the incremental learning may be performed on the learned model. The incremental learning can be performed even after the apparatus is installed at the place of use.

### First Embodiment

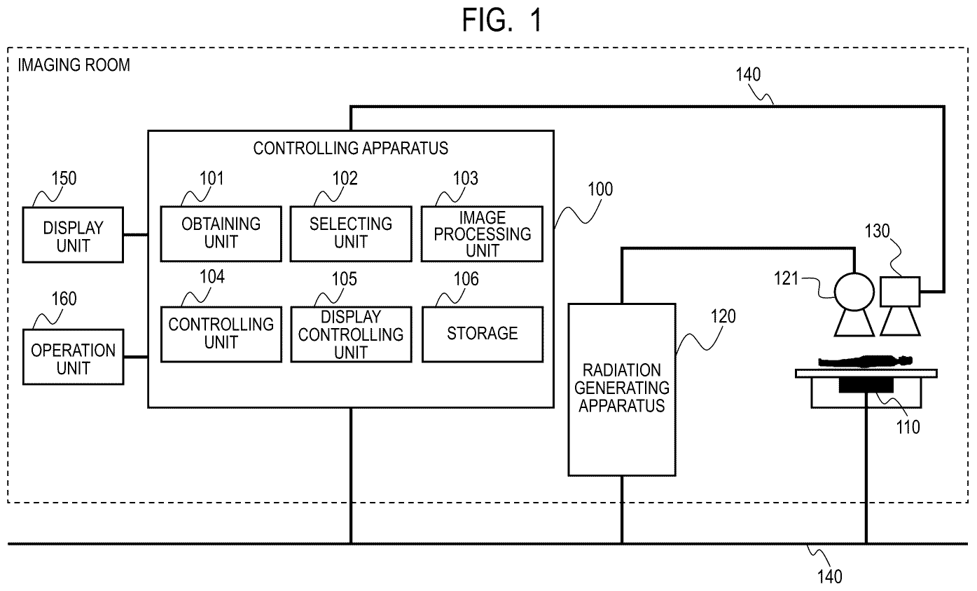

Hereinafter, with reference to FIG. 1 to FIG. 5, a radiation imaging system for imaging and displaying a medical image, an image processing apparatus, and an image processing method according to a first embodiment of the present disclosure will be described.

### <Configuration of Radiation Imaging System>

FIG. 1 is a diagram for illustrating an example of the schematic configuration of the radiation imaging system according to the first embodiment, which is provided in an imaging room. The radiation imaging system according to the first embodiment includes a controlling apparatus 100, a radiation imaging apparatus 110, a radiation generating apparatus 120, a tube bulb 121, a camera 130, and a display unit 150, and an operation unit 160. The controlling apparatus 100, the radiation imaging apparatus 110, the radiation generating apparatus 120, and the camera 130 are connected to each other via any network 140.

The network 140 may include, for example, a LAN (Local Area Network), a WAN (Wide Area Network), or the like. The network 140 may be a wired network or a wireless network. These apparatuses may be directly connected without the network 140. The controlling apparatus 100, the display unit 150, and operation unit 160 may be provided in an operation room partitioned within the imaging room or in an operation room provided separately from the imaging room.

The controlling apparatus 100 can be configured using an information processing apparatus such as a computer, and can function as an example of an image processing apparatus which can control various kinds of imaging and perform image processing of captured images. The computer includes, for example, a main controlling unit such as a CPU and a storage such as a Read Only Memory (ROM), a Random Access Memory (RAM), etc.

The controlling apparatus 100 can communicate with the radiation imaging apparatus 110 to control radiation imaging and obtain a radiation image imaged by the radiation imaging apparatus 110. The controlling apparatus 100 can communicate with the radiation generating apparatus 120 to control the radiation generating apparatus 120 and obtain the information when the radiation is irradiated from the radiation generating apparatus 120. Furthermore, the controlling apparatus 100 can communicate with the camera 130 to control the camera 130 and obtain an image imaged by the camera 130.

The radiation imaging apparatus 110 can transition to an imaging ready state according to an instruction from the controlling apparatus 100, perform the radiation imaging in synchronization with the radiation generating apparatus 120, and generate an image based on the radiation irradiated from the tube bulb 121, which is an example of a radiation source. The radiation imaging apparatus 110 can include any radiation detector that detects the radiation and outputs a corresponding signal, and the radiation detector can be configured using, for example, an FPD (Flat Panel Detector). The radiation detector may be an indirect conversion type detector that temporarily converts the radiation to visible light using a scintillator or the like, and converts the visible light into an electric signal using an optical sensor or the like, or a direct conversion type detector that directly converts the entered radiation into an electric signal. The number of the radiation imaging apparatus 110 is not limited to one, and a plurality of radiation imaging apparatuses may be used.

The radiation generating apparatus 120 is an apparatus that detects a radiation irradiation instruction by a user and generates the radiation from the tube bulb 121 based on irradiation condition set by a user input apparatus (not shown) such as an operation panel, which receives a user operation. The radiation irradiated from the tube bulb 121 transmits the subject with attenuation and enters the radiation imaging apparatus 110.

The camera 130 functions as an example of an optical imaging apparatus that performs imaging according to an instruction from the controlling apparatus 100 and obtains an optical image. In the first embodiment, the camera 130 is attached to the tube bulb 121 and perform the imaging in a radiation generation direction of the tube bulb 121, and has an imaging range equivalent to that of the radiation image. The tube bulb 121 may be an apparatus including the tube bulb itself, which is an example of a radiation source, and the camera 130 may be attached to the apparatus.

The display unit 150 and the operation unit 160 are connected to the controlling apparatus 100. The display unit 150 includes, for example, any display such as a liquid crystal display, and displays various information such as subject information on the subject and the imaging-protocol, and various images such as the radiation image and the optical image under the control of the controlling apparatus 100. The operation unit 160 includes an input device for inputting operation information for operating the controlling apparatus 100, such as a keyboard and a mouse. Note that the display unit 150 may be configured by a touch panel type display, and in this case, the display unit 150 may also be used as the operation unit 160.

Next, the configuration of the controlling apparatus 100 will be described. The controlling apparatus 100 includes an obtaining unit 101, a selecting unit 102, an image processing unit 103, a controlling unit 104, a display controlling unit 105, and a storage 106.

The obtaining unit 101 can obtain the optical image which is obtained by optically imaging the subject by the camera 130 and the radiation image which is obtained by imaging the subject by the radiation imaging apparatus 110 with the radiation. Further, the obtaining unit 101 can obtain the operation information, the subject information, the imaging-protocol related to the imaging, and the like input via the operation unit 160. The obtaining unit 101 may obtain these images, information, and the like from an imaging apparatus or a server (not shown) connected to the controlling apparatus 100 via any network. The obtaining unit 101 may also obtain the images, information, and the like stored in the storage 106.

The selecting unit 102 can select an imaging-protocol related to the imaging of the subject from a plurality of imaging-protocols stored in the storage 106 based on the operation information from the user, which is obtained by the obtaining unit 101. Here, the imaging-protocols may be set, for example, for each imaged site or for each combination of the imaged site, the imaging-condition, and the like. In the first embodiment, an example of imaging-protocol set for each imaged site will be described for simplicity of explanation.

The image processing unit 103 can perform image processing on the optical image and the radiation image obtained by the obtaining unit 101 so that the optical image and the radiation image correspond to each other. The image processing may include, for example, image rotation processing, extraction processing (clipping processing), scaling processing (enlargement/reduction processing), and the like. A state where the optical image and the radiation image correspond to each other is not limited to a state in which the direction, angle, size, etc. of the subject in the optical image and the radiation image are the same, but it may be a state in which the user can easily identify the subject in the optical image and the subject in the radiation image by associating them with each other. Therefore, as long as the user can easily identify the subject in the optical image and the subject in the radiation image by associating them with each other, the direction, angle, size, etc. of the subject in the optical image and the radiation image may be different from each other. Details of the image processing by the image processing unit 103 will be described later.

The controlling unit 104 controls the radiation imaging apparatus 110, the radiation generating apparatus 120, the camera 130, and the network apparatuses (not shown) connected to the network 140. The controlling unit 104 can control the overall operation of the controlling unit 100.

The display controlling unit 105 can control the display of the display unit 150. The display controlling unit 105 can cause, for example, the subject information, the imaging-condition, parameters set by the user, the optical image, the radiation image, the imaging-protocol, and image processing settings to be displayed on the display unit 150. Also, the display controlling unit 105 can cause any displays such as buttons and sliders for receiving user operations, and GUI, etc. to be displayed on the display unit 150, according to the desired configuration.

The storage 106 can store the optical image and the radiation image processed by the controlling apparatus 100, various data, and the like. Further, the storage 106 can store the subject information, the imaging-conditions, parameters set by the user, and the like. Furthermore, the storage 106 can store various control programs for realizing each function of the controlling apparatus 100. The storage 106 may be configured by any storage medium such as an optical disk or memory, for example.

The controlling apparatus 100 may be configured using a general computer or a computer dedicated to the radiation imaging system. The controlling apparatus 100 may be, for example, a personal computer (PC), or a desktop PC, a notebook PC, or a tablet PC (portable information terminal). Furthermore, the controlling apparatus 100 may be configured as a cloud type computer in which some components are arranged in an external apparatus.

The obtaining unit 101, the selecting unit 102, the image processing unit 103, the controlling unit 104, and the display controlling unit 105 may be configured by software modules executed by the processor of the controlling apparatus 100. Furthermore, each of these components may be configured by a circuit or an independent device that performs a specific function such as an ASIC.

Note that the configuration shown in FIG. 1 is only an example, and may be appropriately changed according to a desired configuration. For example, in FIG. 1, various apparatuses are connected to the controlling apparatus 100 via the network 140, but the controlling apparatus 100 need not be connected to such apparatuses, and the controlling apparatus 100 may obtain various images from a server or the like (not shown). Further, for each apparatus, the network has a plurality of apparatuses.

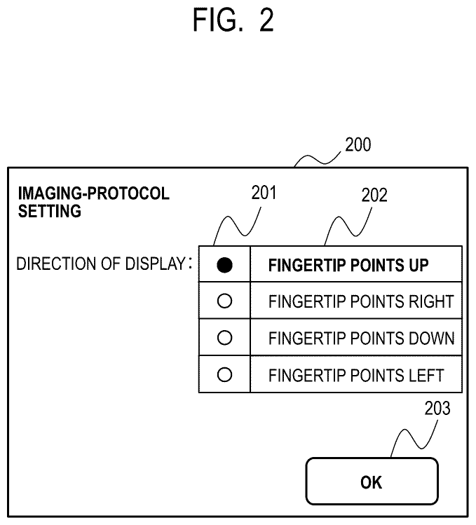

Next, with reference to FIG. 2 to FIG. 5, the imaging preparation processing and the imaging processing according to the first embodiment will be described along the flow of examination by the radiation imaging system according to the first embodiment. In the first embodiment, the imaging preparation processing is performed as a preparation step before the imaging processing. However, it is not necessary to perform the imaging preparation processing for each examination, and it may be omitted as appropriate in a case where the settings of the past examination or the like are used.

### <Imaging Preparation Processing>

First, the imaging preparation processing and the image processing according to the first embodiment will be described with reference to FIG. 2 to FIG. 4. FIG. 2 is a diagram for illustrating an example of an imaging-protocol setting screen 200 according to the first embodiment. The imaging-protocol setting screen 200 can be displayed by the display unit 150 under the control of the display controlling unit 105. In the imaging preparation processing according to the first embodiment, the user operates the operation unit 160 and sets the imaging-protocol on the imaging-protocol setting screen 200 as shown in FIG. 2.

For convenience of explanation, an example of setting an imaging-protocol for a left hand will be described. The imaging-protocol setting screen 200 for the left hand can set the orientation of the subject in the optical image and the radiation image for the display of the optical image and the radiation image on the display unit 150. The user can set the orientation of the subject in the optical image and the radiation image by specifying a radio button 201 corresponding to orientation 202 of the subject to be displayed and pressing the OK button 203 via the operation unit 160. The set imaging-protocol is stored in the storage 106.

In the imaging processing according to the first embodiment, based on imaging-protocol set as described above, the image processing unit 103 performs image processing on the optical image and the radiation image so as to correspond to the set orientation. The image processing according to the first embodiment will be described below.

### <Image Processing of Optical Image>

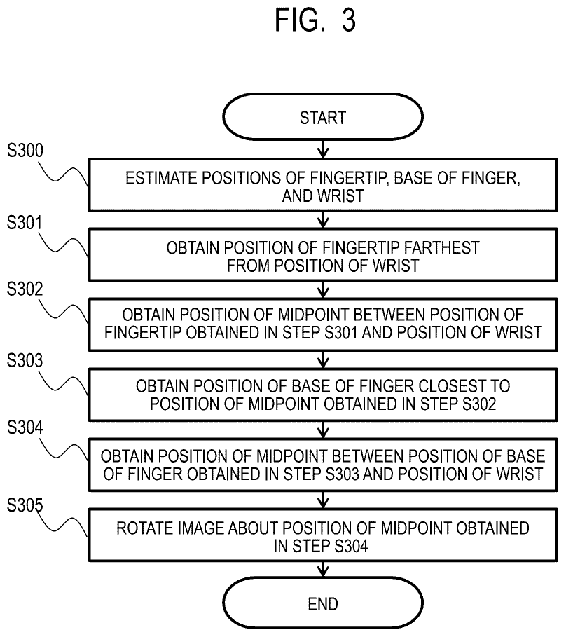

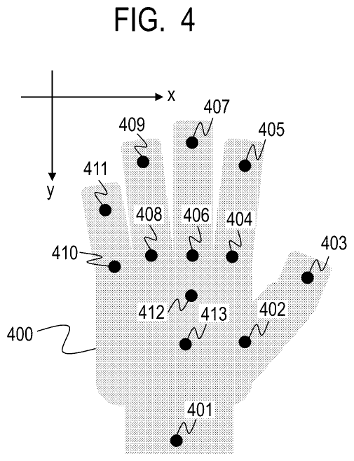

The image processing of the optical image will be described below with reference to FIG. 3 and FIG. 4. FIG. 3 is a flowchart showing an example of the image processing procedure of the optical image according to the first embodiment. In the image processing of the optical image according to the first embodiment, the image processing unit 103 recognizes the orientation of the subject in the optical image, and performs the image processing on the optical image so that the subject in the optical image has the orientation of the subject set in association with the imaging-protocol. An example in which the subject is the left hand in the same manner as the setting of the imaging-protocol will be described below.

First, in step S300, the image processing unit 103 estimates the positions of the fingertip, the base of the finger, and the wrist of the left hand, which are the subjects in the optical image obtained by the camera 130. For example, in a case where the optical image of the left hand 400 as shown in FIG. 4 is obtained, the image processing unit 103 estimates the positions of the fingertips (403, 405, 407, 409, 411), the bases of the fingers (402, 404, 406, 408, 410), and the wrist (401). The coordinate system for these positions is a screen coordinate system, and in the example of FIG. 4, the origin of the coordinate system is the upper left of the screen on which the optical image is displayed. The coordinate system may be freely set according to the desired configuration.

The image processing unit 103 may estimate the positions of the fingertip or the like for the optical image by the rule-based processing based on the regularity of the structure of subject or the like. For example, the image processing unit 103 may perform a well-known edge extraction processing or the like on the optical image to extract feature points, and estimate the positions of the fingertip or the like based on the feature points and the regularity of the structure of the hand or the like.

The image processing unit 103 may also estimate the positions of the fingertip or the like from the optical image using a learned model obtained by the machine learning. The learned model in this case may be obtained by using a training data which includes an optical image as input data and information indicating the positions of the fingertip, the base of the finger, and the wrist in the optical image as output data. The information indicating the positions of the fingertip, the base of the finger, and the wrist in the optical image may be generated by a physician or the like in association with the optical image, and may be generated as coordinates indicating the position of each site or a label image with labels indicating each site in the optical image.

In the case of using the learned model, the controlling apparatus 100 functions as an example of a training unit performing training of the learned model used for the recognition processing of the positions of a fingertip, etc., but the image processing unit 103 may use a learned model trained by another training apparatus or the like. It should be noted that a GPU can perform efficient operation by processing more data in parallel. Therefore, in a case where the training is performed multiple times using a machine learning algorithm such as the deep learning, it is effective to perform the processing by the GPU. Therefore, in the first embodiment, the processing by the controlling apparatus 100 functioning as an example of the training unit may use the GPU in addition to the CPU. Specifically, when executing a training program including a learning model, the training can be performed by the CPU and the GPU cooperatively performing the operations. In the processing of the training unit, the calculation may be performed only by the CPU or the GPU. Further, the estimation processing according to the first embodiment may be implemented using the GPU as in the case of the learning unit. In a case where the learned model is provided in an external apparatus, the controlling apparatus 100 need not function as the training unit.

The training unit may also include an error detecting unit and an updating unit (not shown). The error detecting unit obtains an error between output data output from the output layer of the neural network in accordance with input data input to the input layer, and the ground truth. The error detecting unit may calculate the error between the output data from the neural network and the ground truth using a loss function. Further, based on the error obtained by the error detecting unit, the updating unit updates combining weighting factors between nodes of the neural network or the like so that the error becomes small. The updating unit updates the combining weighting factors or the like by using, for example, the error back-propagation method. The error back-propagation method is a method that adjusts the combining weighting factors between the nodes of each neural network or the like so that the above error becomes small.

As the machine learning model according to the first embodiment, for example, FCN (Fully Convolutional Network) or SegNet can be used. As the machine learning model for object recognition, for example, RCNN (Region CNN), fastRCNN, or fasterRCNN can be used. Furthermore, YOLO (You Only Look Once) or SSD (Single Shot Detector or Single Shot MultiBox Detector) may be used as the machine learning model for the object recognition in units of regions.

Next, in step S301, the image processing unit 103 obtains the position of the fingertip farthest from the position of the wrist 401. Specifically, the image processing unit 103 obtains the distances between the fingertips 403, 405, 407, 409, 411 and the wrist 401, and obtains the position of the fingertip with the largest value. In the example shown in FIG. 4, the image processing unit 103 obtains the position of the fingertip 407 as the position of the fingertip farthest from the position of the wrist 401.

In step S302, the image processing unit 103 obtains the position of the midpoint between the position of the fingertip 407 obtained in step S301 and the position of the wrist 401. Specifically, the image processing unit 103 adds the X coordinate of the wrist 401 and the X coordinate of the fingertip 407, and divides the addition result by 2 to obtain the midpoint X coordinate. Further, the image processing unit 103 adds the Y coordinate of the wrist 401 and the Y coordinate of the fingertip 407, and divides the addition result by 2 to obtain the midpoint Y coordinate. In the example shown in FIG. 4, the image processing unit 103 obtains the position of the midpoint 412 as the midpoint between the position of the fingertip 407 obtained in step S301 and the position of the wrist 401.

In step S303, the image processing unit 103 obtains the nearest position of the base of the finger from the position of the midpoint 412 obtained in step S302. Specifically, the image processing unit 103 obtains the distances between the midpoint 412 and the bases of the fingers 402, 404, 406, 408, 410, and obtains the position of the base of the finger with the smallest distance value. In the example shown in FIG. 4, the image processing unit 103 obtains the position of the base of the finger 406 as the closest position of the base of the finger from the position of the midpoint 412 obtained in step S302.

In step S304, the image processing unit 103 obtains the position of the midpoint between the position of the base of the finger 406 obtained in step S303 and the position of the wrist 401. Specifically, the image processing unit 103 adds the X coordinate of the wrist 401 and the X coordinate of the base of the finger 406, and divides the addition result by 2 to obtain the X coordinate of the midpoint. Further, the image processing unit 103 adds the Y coordinate of the wrist 401 and the Y coordinate of the base of the finger 406, and divides the addition result by 2 to obtain the Y coordinate of the midpoint. In the example shown in FIG. 4, the image processing unit 103 obtains the position of the midpoint 413 as the position of the midpoint between the position of the base of the finger 406 obtained in step S303 and the position of the wrist 401.

In step S305, the optical image is rotated around the position of the midpoint 413 obtained in step S304 in accordance with the orientation of the subject set in the imaging preparation processing described above. When rotating the optical image, the orientation of the subject in which the Y-coordinate of the fingertip 407 obtained in step S301 is closest to 0 (smallest) can be set to the orientation of “finger points up”, and the orientation of the subject in which the Y-coordinate is largest can be set to the orientation of “finger points down”. In addition, the orientation of the subject in which the X-coordinate of the fingertip 407 obtained in step S301 is closest to 0 (smallest) can be set to the orientations of “finger points left”, and the orientation of the subject in which the X-coordinate is largest can be set to the orientation of “finger points right”. When the processing in step S305 is completed, the image processing unit 103 ends the image processing.

### <Image Processing of Radiation Image>

The image processing of the radiation image according to the first embodiment may be the same as the image processing of the optical image. Therefore, except that the processing target changes from the optical image to the radiation image, the image processing unit 103 performs the same processing as the processing in steps S300 to S305 on the radiation image. More specifically, the image processing unit 103 recognizes the orientation of the subject in the radiation image and performs the image processing on the radiation image so that the subject in the radiation image has the orientation of the subject set in relation to the imaging-protocol. Since the details of each step are the same as those in steps S300 to S305, the description thereof is omitted.

A learned model in the case where the image processing unit 103 estimates the positions of the fingertip or the like from the radiation image using a learned model obtained by the machine learning can be generated using a radiation image as training data. Specifically, the learned model in this case may be obtained using training data which includes a radiation image as input data and information indicating the positions of the fingertip, the base of the finger, and the wrist in the radiation image as output data. The information indicating the positions of the fingertip, the base of the finger, and the wrist in radiation image may be generated by a physician or the like in association with the radiation image, and may be generated as coordinates indicating the position of each site or a label image with labels indicating each site in the radiation image. Note that even in this case, the controlling apparatus 100 functions as an example of the training unit performing training of a learned model for estimating the positions of the fingertip or the like of the subject, but the image processing unit 103 may use a learned model trained by another training apparatus or the like.

Note that the specific method of processing for estimating and recognizing the orientation of the subjects according to the first embodiment is not limited to the above method, and any known method may be used. For example, the image processing unit 103 may recognize the orientation of the subject from the optical image using a learned model obtained using training data which include an optical image as input data and information indicating the orientation of the subject in the optical image as output data. Similarly, the image processing unit 103 may recognize the orientations of the subject from the radiation image using a learned model obtained using training data which includes a radiation image as input data and information indicating the orientation of the subject in the radiation image as output data. Even in these cases, the controlling apparatus 100 functions as an example of a training unit performing training of a learned model for recognizing the orientation of the subject, but the image processing unit 103 may use a learned model trained by another learning apparatus or the like.

The method for determining the rotation axis of the image is not limited to the above method. For example, the image processing unit 103 may determine the center of the image as the rotation axis of the image.

The image processing may be processing for identifying the orientation of the subject and rotating the image so that the orientation of the subject in the image becomes the specified orientation, processing specifying the rotation angle numerically in advance and rotating the image, or processing for extracting a part of the image. The image processing may include processing for enlarging or reducing the image.

The image processing to be performed on the optical image and the radiation image need not be the same. For example, the rotation processing and the enlargement processing may be set as the image processing for the optical image, and only the rotation processing may be set as the image processing for the radiation image. However, the image processing for the optical image and the image processing for the radiation image may be such that the appearances of the optical image and the radiation image on which the image processing is performed correspond to each other.

### <Imaging Processing>

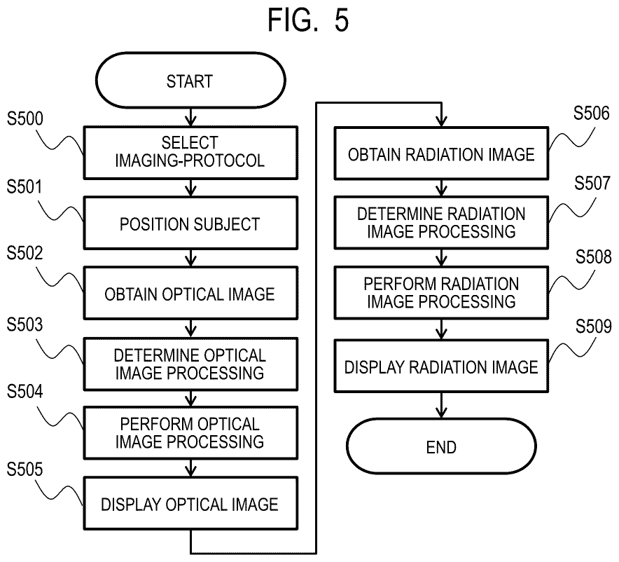

The procedure for performing the imaging processing according to the first embodiment will be described below with reference to FIG. 5. FIG. 5 is a flowchart showing an example of the procedure of the imaging processing according to the first embodiment. First, in step S500, the obtaining unit 101 obtains operation information from the user input via the operation unit 160, and selects the imaging-protocol based on the operation information. An example in which the imaging-protocol for left hand is selected will be described below. In the first embodiment, when the imaging-protocol is selected, the examination of the subject is started.

Next, in step S501, the user positions the subject so that the left hand of the patient, which is the subject, is imaged in the optical image and the radiation image. The positioning of the subject may be performed in an imaging position such as standing, lying or sitting position according to the desired imaging-condition.

In step S502, the controlling unit 104 controls the imaging using the camera 130, and the obtaining unit 101 obtains an optical image including the subject from the camera 130. In this example of explanation, the obtaining unit 101 obtains the optical image in which the left hand of the positioned patient is imaged.

In step S503, the image processing unit 103 determines image processing to be performed on the optical image based on the imaging-protocol selected in step S500. Specifically, the image processing unit 103 determines the image processing based on the imaging-protocol set in the imaging preparation processing stored in the storage 106 as the image processing to be performed for the optical image. In this example of explanation, the image processing unit 103 determines image processing for rotating the optical image so that the fingertip of the left hand in the optical image points up based on the setting of imaging-protocol for the left hand.

In step S504, the image processing unit 103 applies the image processing determined in step S503 to the optical image obtained in step S502. In this example of explanation, the image processing unit 103 performs the image processing for rotating the optical image so that the fingertip of the left hand in the optical image points up according to the procedure described in steps S300 to S305.

In step S505, the display controlling unit 105 causes the display unit 150 to display the image processed the optical image. Thus, the user can check whether there is any problem with the positioning of subject. If there is no problem with the positioning of subject, the user can input instruction of the radiation imaging into the controlling apparatus 100 by pressing a radiation irradiation switch (not shown) or the like.

In step S506, the controlling unit 104 controls the radiation generating apparatus 120 according to the input instruction of the radiation imaging to irradiate the radiation toward the subject by the tube bulb 121, and detects the radiation that has passed through the subject by the radiation imaging apparatus 110. Then, the obtaining unit 101 obtains a radiation image including the subject imaged by the radiation imaging apparatus 110. In this example of explanation, the obtaining unit 101 obtains the radiation image imaging the left hand of the patient.

In step S507, similarly to step S503, the image processing unit 103 determines the image processing to be performed on the radiation image based on the imaging-protocol selected in step S500. Specifically, the image processing unit 103 determines the image processing based on the imaging-protocol set in the imaging preparation processing stored in the storage 106 as the image processing to be performed on the radiation image. In this example of explanation, the image processing unit 103 determines image processing for rotating the radiation image so that the fingertip of the left hand in the radiation image points up based on the setting of the imaging-protocol for the left hand.

In step S508, the image processing unit 103 applies the image processing determined in step S507 to the radiation image obtained in step S506. In this example of explanation, the image processing unit 103 performs the image processing to rotate radiation image so that the fingertip of the left hand in radiation image faces upward by the same procedure as described in steps S300 to S305.

In step S509, the display controlling unit 105 causes the display unit 150 to display the radiation image subjected to the image processing. Thus, the user can check the optical image and the radiation image in the manner in which the optical image and the radiation image correspond to each other, and can easily judge whether or not the positioning of the subject was properly performed in the radiation imaging. When the processing in step S509 is completed, the controlling apparatus 100 ends the imaging processing

The imaging-protocol for the left hand is described as an example, but an imaging-protocol to be selected is not limited thereto. For example, an imaging-protocol for right hand, left foot, right foot, chest, head, abdomen, or the like may be selected. For these imaging-protocol, the orientation of the subject in the optical image and the radiation image when the optical image and the radiation image are displayed on display unit 150 can be set by the imaging preparation processing. The learned model for recognizing the orientation of the subject or the site of the subject may be provided for each imaged site or imaging-protocol. In this case, the image processing unit 103 can select a learned model in accordance with the imaged site or the imaging-protocol and use it for the image processing.

As described above, the radiation imaging system according to the first embodiment includes the camera 130 that functions as an example of an optical imaging apparatus for imaging subject optically, the radiation imaging apparatus 110 for imaging the subject with radiation, and the controlling apparatus 100 that functions as an example of an image processing apparatus. The controlling apparatus 100 includes the obtaining unit 101 and the image processing unit 103. The obtaining unit 101 obtains an optical image which is obtained by imaging the subject optically and a radiation image which is obtained by imaging the subject with the radiation. The image processing unit 103 performs image processing including at least one of rotation processing, extraction processing, and scaling processing on the optical image and the radiation image so that the optical image and radiation image correspond to each other.

With this configuration, the controlling apparatus 100 according to the first embodiment can perform the image processing on the optical image and the radiation image so that the optical image and the radiation image correspond to each other. Therefore, the controlling apparatus 100 can make the appearances of the optical image and the radiation image correspond to each other, provide images that are easy for the user to check, reduce the need to ask the patient to move, reduce the need to adjust the image, and reduce the burden on the patient and the user.

Further, the controlling apparatus 100 may include an selecting unit 102 that selects an imaging-protocol for the imaging of the subject. The image processing unit 103 may perform the image processing corresponding to the imaging-protocol for the optical image and the radiation image so that the optical image and the radiation image correspond to each other. In this case, by performing the image processing corresponding to the imaging-protocol, the controlling apparatus 100 can perform the image processing corresponding to the purpose and the procedure of the imaging suitably, and provide an image that is easier for the user to check.

Furthermore, the image processing unit 103 may recognize the orientation of subject in the optical image and perform the image processing on the optical image so that subject in the optical image has a predetermined orientation. The image processing unit 103 may recognize the direction of the subject in the radiation image and perform the image processing on the radiation image so that the subject in the radiation image has a predetermined orientation. With this configuration, the controlling apparatus 100 can perform the image processing so that the subject in the image has a desired orientation regardless of the orientation of the patient. Therefore, the controlling apparatus 100 can reduce the need for the patient to move or the need for work such as image adjustment, which is caused by the difficulty in checking the optical image when positioning the subject, thereby reducing the burden on the patient and the user. Furthermore, the controlling apparatus 100 can perform the image processing so that subject has the desired orientation, thereby improving the convenience of the radiation imaging system.

Furthermore, in a case where the subject is a human hand, the image processing unit 103 may estimate positions of a fingertip and a wrist of the subject in the optical image and the radiation image. In this case, the image processing unit 103 can recognize the orientation of the subject in the optical image based on the positions of the fingertip and wrist of the subject in the optical image. Furthermore, image processing unit 103 can recognize the orientation of the subject in the radiation image based on the positions of the fingertip and wrist of the subject in the radiation image. With this configuration, the image processing unit 103 can recognize the orientation of the subject in the optical image or the radiation image.

Furthermore, in a case where the subject is a human hand, the image processing unit 103 may further estimate the position of the base of finger of the subject in the optical image and the radiation image. In this case, the image processing unit 103 may obtain a rotation axis of the optical image based on the positions of the fingertip, the base of the finger, and the wrist of the subject in the optical image, and may perform the rotation processing on the optical image around the rotation axis. Further, the image processing unit 103 may obtain a rotation axis of the radiation image based on the positions of the fingertip, the base of the finger, and the wrist of the subject in the radiation image, and may perform the rotation processing on the radiation image around the rotation axis. According to such a configuration, the controlling apparatus 100 can determine a more appropriate rotation axis for the image showing the hand and perform the rotation processing.

Further, the image processing unit 103 may recognize the orientation of the subject in the obtained optical image by using an output from a learned model obtained by inputting the obtained optical image into the learned model which has been obtained by using training data including an optical image and information indicating an orientation of a subject in the optical image. Furthermore, the image processing unit 103 may recognize the orientation of the subject in the obtained radiation image by using an output from a learned model obtained by inputting the obtained radiation image into the learned model which has been obtained by using training data including a radiation image and information indicating an orientation of a subject in the radiation image. Even with such a configuration, the image processing unit 103 can recognize the orientation of the subject in the optical image or the radiation image.

In the first embodiment, the obtaining unit 101 obtains the optical image and the radiation image. On the other hand, an optical image obtaining unit for obtaining an optical image and a radiation image obtaining unit for obtaining a radiation image may be provided separately. In this case, the obtaining unit 101 may be provided as a component including the optical image obtaining unit and the radiation image obtaining unit. Similarly, the image processing unit 103 performs the image processing on the optical image and the radiation image. On the other hand, an optical image processing unit for performing image processing on the optical image and a radiation image processing unit for performing image processing on the radiation image may be provided separately. In this case, the image processing unit 103 may be provided as a component including the optical image processing unit and the radiation image processing unit.

In the first embodiment, the obtaining unit 101 obtains the radiation image and the optical image from the radiation imaging apparatus 110 and the camera 130. On the other hand, the obtaining unit 101 may obtain these images and the like from an imaging apparatus or a server (not shown) connected to the controlling apparatus 100 via any network. In this case, the obtainment time of the radiation image and the optical image may be earlier or later as long as the subject takes the same imaging posture.

In the first embodiment, the image processing unit 103 performs the rotation processing of the optical image and the radiation image based on the setting of the orientation of the subject associated with the imaging-protocol. On the other hand, the image processing unit 103 may perform image processing on at least one of the optical image and the radiation image such that one of the orientation of the subject in optical image and the orientation of the subject in the radiation image has the orientation of the other. The other image may be an image on which no image processing has been performed, or an image on which image processing such as rotation processing has been performed based on the setting of the orientation of subject associated with the imaging-protocol.

For example, the image processing unit 103 may recognize the orientation of the subject in the optical image and the radiation image, and may perform image processing such as rotation processing on the radiation image so that the orientation of subject in the radiation image has the orientation of the subject in the optical image. Similarly, the image processing unit 103 may recognize the orientation of the subject in the optical image and the radiation image, and may perform image processing such as rotation processing for the optical image so that the orientation of the subject in the optical image has the orientation of the subject in the radiation image. In this case, the image processing of the optical image and the display processing of the optical image after the image processing may be performed after the obtainment of the radiation image.

Even in the above-described configuration, the controlling apparatus 100 can correspond to the appearances of the optical image and the radiation image, provide an image that is easy for the user to check, reduce the need for the patient to move, reduce the need for the work of the image adjustment, and reduce the burden on the patient and the user. The method for recognizing the orientation of the subject in the optical image and the radiation image may be the same as the method described above.

### Second Embodiment

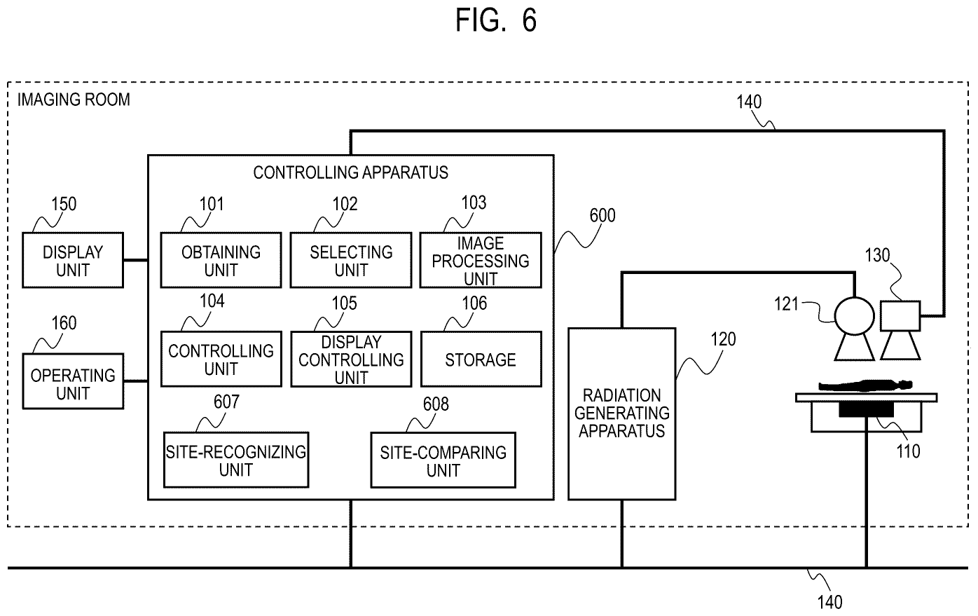

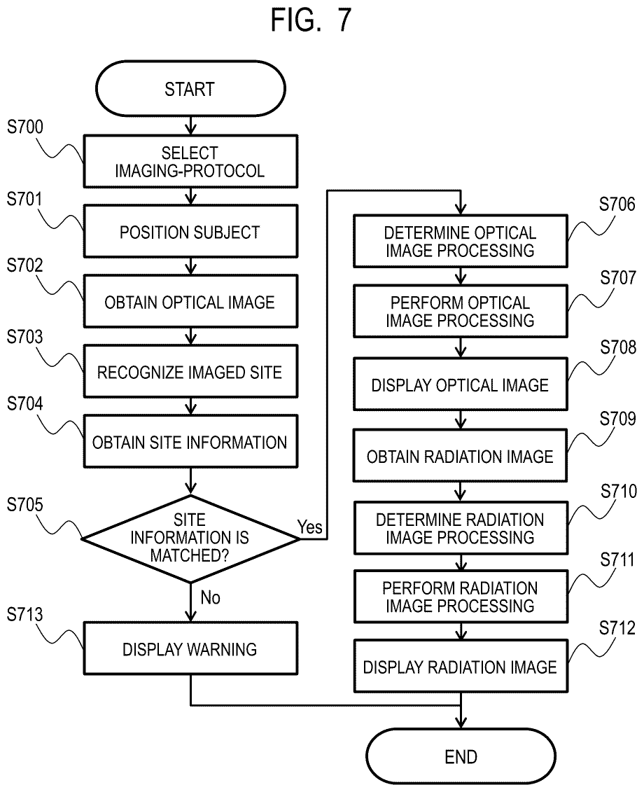

In the first embodiment, the image processing is always applied to the optical image and the radiation image. In contrast, in a second embodiment of the present disclosure, an example in which image processing is performed based on a specific condition will be described. A radiation imaging system, an image processing apparatus and an image processing method according to the second embodiment will be described below with reference to FIG. 6 and FIG. 7. The same configuration, functions and operations as those of the first embodiment will be omitted, and differences between the second embodiment and the first embodiment will be mainly described.

### <Radiation Imaging System Configuration>

FIG. 6 is a diagram for illustrating an example of the schematic configuration of the radiation imaging system according to the second embodiment. The controlling apparatus 600 according to the second embodiment includes a site recognizing unit 607 and a site comparing unit 608 in addition to the configuration of the controlling apparatus 100 according to the first embodiment.

The site recognizing unit 607 recognizes an imaged site in the optical image obtained by the camera 130. The site recognizing unit 607 may recognize the imaged site in the optical image by any known method. For example, the site recognizing unit 607 may recognize the imaged site in the optical image by rule-based processing based on the regularity of the structure of the subject or the like. In this case, the site recognizing unit 607 may extract feature point by performing, for example, a well-known edge extraction processing on the optical image, and recognize the imaged site based on feature point and the regularity of the structure of each imaged site or the like.

The site recognizing unit 607 may recognize the imaged site in the optical image by using a learned model obtained by the machine learning. The learned model in this case may be obtained by using training data which includes an optical image as input data and information indicating an imaged site in the optical image as output data. The information indicating the imaged site in the optical image may be generated by a physician or the like in association with the optical image. In the case where the learned model is used, the controlling apparatus 600 functions as an example of a training unit performing training of the learned model used for recognition processing of the imaged site, but the site recognizing unit 607 may use a learned model trained by another training apparatus or the like.

The site comparing unit 608 compares the imaged site recognized by the site recognizing unit 607 with site information indicating an imaged site associated with the imaging-protocol.

### <Imaging Processing>

Next, the procedure of the imaging processing according to the second embodiment will be described with reference to FIG. 7. FIG. 7 is a flowchart showing an example of the procedure of the imaging processing according to the second embodiment. Since the imaging preparation processing according to the second embodiment is the same as the imaging preparation processing according to the first embodiment, the description thereof will be omitted.

Since the processing of steps S700 to S702 according to the second embodiment is the same as the processing of steps S500 to S502 according to the first embodiment, the description thereof will be omitted. If the obtaining unit 101 obtains the optical image in step S702, the process shifts to step S703. In step S703, the site recognizing unit 607 recognizes an imaged site of the subject in the optical image, and outputs the recognition result to the site comparing unit 608.

In step S704, the site comparing unit 608 obtains site information associated with the imaging-protocol selected in step S700 from the storage 106. The site comparing unit 608 may obtain the site information associated with the imaging-protocol from an external storage apparatus such as a server (not shown) connected to the controlling apparatus 600.

In step S705, the site comparing unit 608 compares the imaged site recognized in step S703 with the site information obtained in step S704, and judges whether or not they match. In step S705, if it is judged that the imaged site and the site information match, the process shifts to step S706. Since the processes of steps S706 to S712 are the same as those of steps S503 to S509 according to the first embodiment, description thereof is omitted.

On the other hand, if it is judged that the imaged site and the site information do not match in step S705, the process shifts to step S713. In step S713, the display controlling unit 105 causes the display unit 150 to display a warning such as a dialog box informing that the imaged site and the site information of the imaging-protocol do not match. The warning may include, for example, a message urging correction of the posture of the subject or change of the imaged site. If the warning is displayed in step S713, the imaging processing according to the second embodiment ends. Therefore, in the second embodiment, if the imaged site and the site information do not match, the imaging processing ends without performing the image processing on the optical image by the image processing unit 103. In this case, the imaging processing ends without obtaining the radiation image by the obtaining unit 101.

As described above, the controlling apparatus 600 according to the second embodiment includes the site recognizing unit 607 and the site comparing unit 608. The site recognizing unit 607 functions as an example of a recognizing unit that recognizes an imaged site of the subject from the optical image. The site comparing unit 608 functions as an example of a judging unit that judges whether the recognized imaged site matches with site information indicating an imaged site of the subject associated with the imaging-protocol. In a case where it is judged that the recognized imaged site does not match with the site information, the image processing unit 103 does not perform the image processing on the optical image.

With this configuration, the controlling apparatus 600 can reduce the processing load by not performing the image processing in a case where the imaged site in the optical image is different from an imaged site which is an imaging-target. Further, in the case where it is judged that the recognized imaged site does not match with the site information, the obtaining unit 101 does not obtain the radiation image. According to this configuration, if the imaged site in the optical image is different from an imaged site which is an imaging-target, unnecessary exposure of the subject to the radiation can be prevented by not performing the radiation imaging.

Moreover, the controlling apparatus 600 further includes the display controlling unit 105 for causing a warning to be displayed on the display unit 150 in the case where it is judged that the recognized imaged site does not match with the site information. In this case, the user can perform the position alignment of the subject again based on the warning, thereby improving the workflow.

The site recognizing unit 607 may recognize the imaged-site of the subject in the obtained optical image by using an output from a learned model obtained by inputting the obtained optical image into the learned model which has been obtained by using training data including an optical image and information indicating an imaged site of a subject in the optical image. In this case, the controlling apparatus 600 can obtain a highly accurate recognition result, and can perform a more accurate imaged site determination process.

Regarding the recognition processing of the imaged site, it is expected that the accuracy of the recognition processing can be enhanced by increasing the number of feature parts included in the optical image. Therefore, in order to improve the accuracy of the recognition processing of the imaged site, the site recognizing unit 607 can use an optical image in which the whole image of the subject is imaged. On the other hand, in a case where the user checks the optical image, an optical image focusing on the imaged site may be desired. Therefore, separate optical image may be used for the optical image to be used by the site recognizing unit 607 and the optical image to be processed by the image processing unit 103 and caused to be displayed on display unit 150 by display controlling unit 105. In addition, the image processing unit 103 may perform image processing to extract a region of the imaged site in the optical image from the optical image used by the site recognizing unit 607.

### Third Embodiment

In the first embodiment, the image processing is performed on the optical image and the radiation image based on the analysis results of the images obtained by the camera 130 and the radiation imaging apparatus 110. On the other hand, in a third embodiment of the present disclosure, an example in which image processing is performed on the optical image and the radiation image so that the appearances of the images become preset appearances without analyzing the image will be described. In the third embodiment, the optical imaging apparatus is maintained in the same state from the imaging preparation processing to the imaging processing. On the other hand, the radiation imaging apparatus may be arranged with a different angle between the imaging preparation processing and the imaging processing, and the radiation image is subjected to the image processing based on angle information of the radiation imaging apparatus.

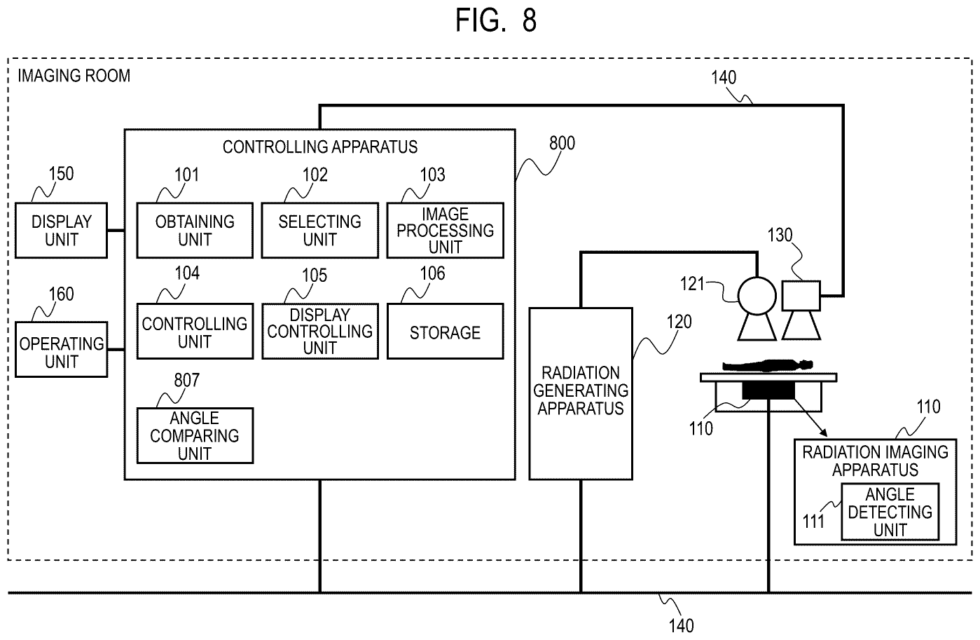

A radiation imaging system, an image processing apparatus, and an image processing method according to the third embodiment will be described below with reference to FIG. 8 to FIG. 13. The same configuration, functions, and operations as those of the first embodiment will be omitted, and differences between the first embodiment and the first embodiment will be mainly described.

### <Radiation Imaging System Configuration>

FIG. 8 is a diagram for illustrating an example of the schematic configuration of a radiation imaging system according to the third embodiment. The radiation imaging apparatus 110 according to the third embodiment additionally includes an angle detecting unit 111. The angle detecting unit 111 can measure the roll, pitch, and yaw angles of the radiation imaging apparatus 110, and can transmit the measurement result to the controlling apparatus 800 or the like via the network 140. The angle detecting unit 111 may include an angle sensor, an acceleration sensor, or other means. The angle detecting unit 111 may also include a combination of these.

The controlling apparatus 800 according to the third embodiment includes an angle comparing unit 807 in addition to the configuration of the controlling apparatus 100 according to the first embodiment. The angle comparing unit 807 can compare the detected angle detected by the angle detecting unit 111 with a set angle of the radiation imaging apparatus 110 associated with an imaging-protocol.

### <Imaging Preparation Processing>

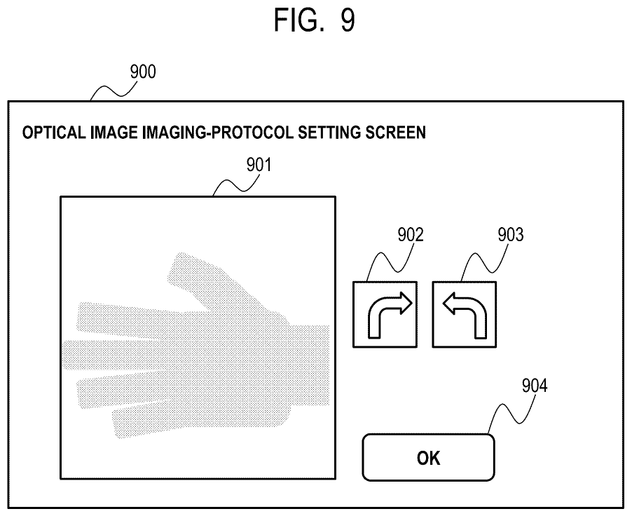

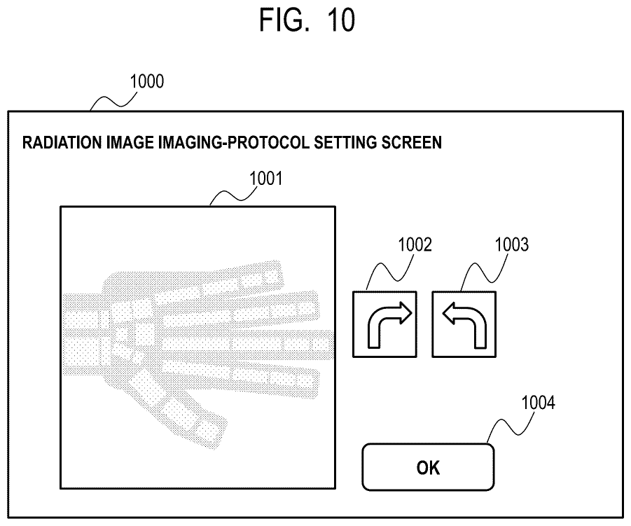

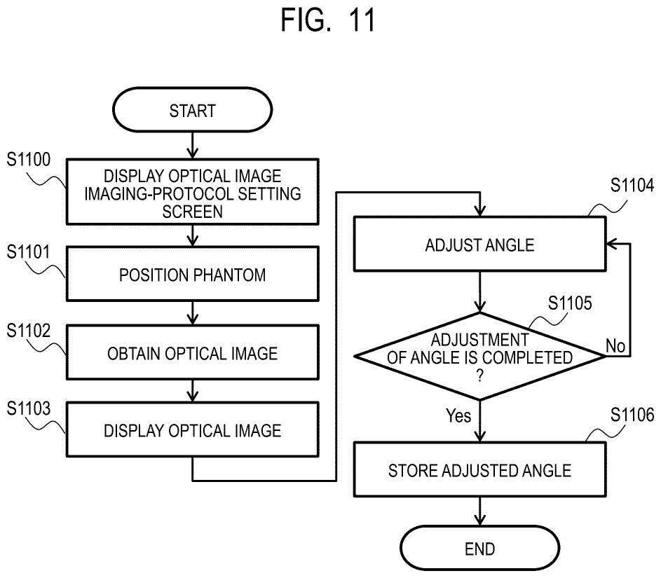

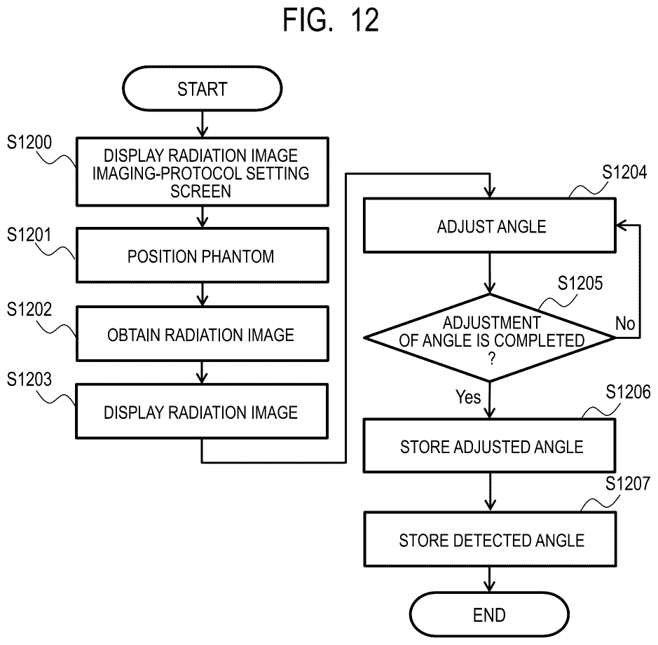

FIG. 9 is a diagram for illustrating an example of a setting screen of the imaging-protocol for the optical image according to the third embodiment. FIG. 10 is a diagram for illustrating an example of a setting screen of the imaging-protocol for the radiation image according to the third embodiment. FIG. 11 is a flowchart showing an example of the procedure of the imaging preparation processing for the optical image according to the third embodiment. FIG. 12 is a flowchart showing an example of the procedure of the imaging preparation processing for the radiation image according to the third embodiment. The imaging preparation processing according to the third embodiment will be described below with reference to each setting screen and flowchart.

First, with reference to FIG. 9 and FIG. 11, the imaging preparation processing for setting the imaging-protocol of the optical image will be described. In step S1100, the obtaining unit 101 obtains the operation information by the user input via the operation unit 160, and the display controlling unit 105 causes the display unit 150 to display an imaging-protocol setting screen 900 of the optical image based on the operation information. Here, for convenience of explanation, the setting of the imaging-protocol for the left hand will be described as an example. On the imaging-protocol setting screen 900 of the optical image, the image processing to be applied to the optical image obtained using the camera 130 can be set in advance when the imaging-protocol is selected and the examination is started.

In step S1101, the user positions a phantom (model) of a subject so that the phantom is imaged on an optical image. In this example of explanation, the user positions a phantom of the left hand.

In step S1102, the controlling unit 104 controls the imaging by the camera 130, and the obtaining unit 101 obtains an optical image including the phantom from the camera 130. In this example of explanation, the obtaining unit 101 obtains an optical image obtained by imaging the positioned phantom of the left hand.

In step S1103, the display controlling unit 105 causes the display unit 150 to display the obtained optical image. At this time, the display controlling unit 105 causes the obtained optical image to be displayed on an optical image display area 901 in the imaging-protocol setting screen 900.

In step S1104, the user checks the optical image displayed on the optical image display area 901, and while viewing the image of the phantom in the optical image, presses a right rotation button 902 or a left rotation button 903 so that the desired display is achieved. The obtaining unit 101 obtains operation information by the user, and the image processing unit 103 performs rotation processing on optical image based on the operation information to adjust the angle of the optical image. The display controlling unit 105 causes an optical image which is updated as needed based on the operation information to be displayed on the optical image display area 901.

In step S1105, the controlling apparatus 800 judges whether or not the angle adjustment is complete. If the operation information indicating that an OK button 904 is pressed is obtained by the obtaining unit 101, the controlling apparatus 800 judges that the angle adjustment is complete. If it is judged that the angle adjustment is complete in step S1105, the process shifts to step S1106. On the other hand, if the operation information indicating that the OK button 904 is pressed is not obtained by the obtaining unit 101 in step S1105, the controlling apparatus 800 judges that the angle adjustment is not complete and returns the process to step S1104.

In step S1106, the controlling apparatus 800 stores the rotation angle of the optical image when the angle adjustment is complete in the storage 106, in association with the imaging-protocol. When the process in step S1106 is completed, the controlling apparatus 800 ends the setting processing of the imaging-protocol of the optical image as the imaging preparation processing.

Next, with reference to FIG. 10 and FIG. 12, the imaging preparation processing for setting an imaging-protocol of the radiation image will be described. In step S1200, the obtaining unit 101 obtains operation information by the user input via the operation unit 160, and the display controlling unit 105 causes the display unit 150 to display an imaging-protocol setting screen 1000 of the radiation image based on the operation information. Here, for convenience of explanation, the setting of imaging-protocol for the left hand will be described as an example. On the imaging-protocol setting screen 1000 of the radiation image, the image processing to be applied to the radiation image obtained using the radiation imaging apparatus 110 can be set in advance when the imaging-protocol is selected and examination is started. The set radiation image processing can be stored in the storage 106 in association with the above-described optical image processing.

In step S1201, the user positions the phantom of the subject so that the phantom is imaged on a radiation image. In this example of explanation, the user positions the phantom of the left hand.

In step S1202, the controlling unit 104 controls the radiation generating apparatus 120 in accordance with the input instruction of radiation imaging to irradiate the radiation toward the subject by the tube bulb 121, and controls the radiation imaging apparatus 110 to detect the radiation transmitted through the subject. Subsequently, the obtaining unit 101 obtains the radiation image including the phantom imaged by the radiation imaging apparatus 110. In this example of explanation, the obtaining unit 101 obtains radiation image obtained by imaging the phantom of the left hand.

In step S1203, the display controlling unit 105 causes the display unit 150 to display the obtained radiation image. At this time, the display controlling unit 105 causes the obtained radiation image to be displayed on the radiation image display area 1001 in the imaging-protocol setting screen 1000.

In step S1204, the user checks the radiation image displayed on the radiation image display area 1001, and while viewing the image of the phantom in the radiation image, presses a right rotation button 1002 or a left rotation button 1003 so that the desired display is achieved. The obtaining unit 101 obtains operation information by the user, and the image processing unit 103 performs rotation processing on the radiation image based on the operation information to adjust the angle of the radiation image. The display controlling unit 105 causes a radiation image which is updated as needed based on the operation information to be displayed on the display area 1001.

In step S1205, the controlling apparatus 800 judges whether or not the angle adjustment is complete. If the operation information indicating that an OK button 1004 is pressed is obtained by obtaining unit 101, the controlling apparatus 800 judges that the angle adjustment is complete. If it is judged that the angle adjustment is complete in step S1205, the process shifts to step S1206. On the other hand, if the obtaining unit 101 does not obtain the operation information indicating that the OK button 1004 is pressed in step S1205, the controlling apparatus 800 judges that the angle adjustment has not been completed and returns the process to step S1204.

In step S1206, the controlling apparatus 800 stores the rotation angle of the radiation image when the angle adjustment is completed in the storage 106, in association with the imaging-protocol. Thus, the image processing of the radiation image can be stored in the storage 106 in association with the image processing of the optical image.

In step S1207, the angle detecting unit 111 stores the angle (yaw, pitch, and roll angles) of the radiation imaging apparatus 110 detected when the angle adjustment is completed in the storage 106 in association with the imaging-protocol. When the processing in step S1207 is completed, the controlling apparatus 800 ends the setting processing of the imaging-protocol of the radiation image as the imaging preparation processing.

### <Imaging Processing>

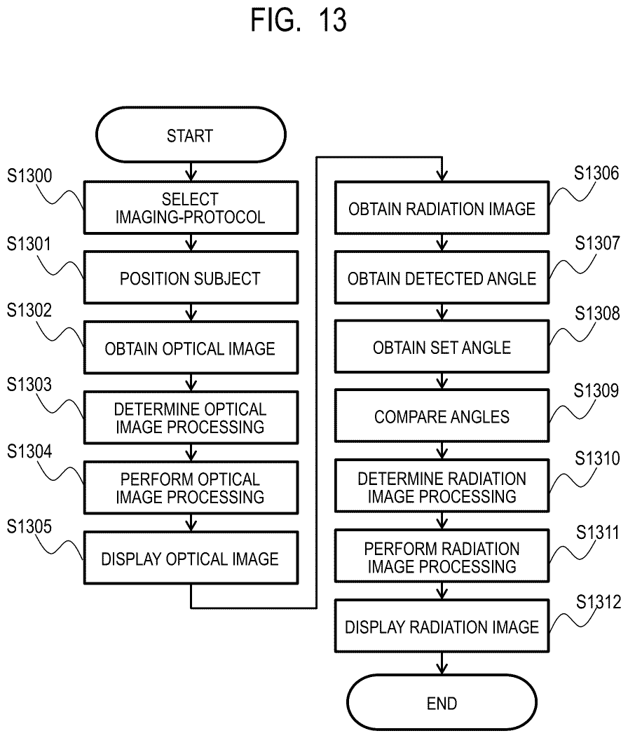

Next, the procedure of the imaging processing according to the third embodiment will be described with reference to FIG. 13. FIG. 13 is a flowchart showing an example of the imaging processing procedure of the third embodiment.

Since the processes of steps S1300 to S1302 according to the third embodiment are the same as the processes of steps S500 to S502 according to the first embodiment, the description thereof is omitted. If the obtaining unit 101 obtains the optical image in step S1302, the process shifts to step S1303.

In step S1303, the image processing unit 103 determines image processing to be performed on the optical image based on the imaging-protocol selected in step S1300. Specifically, the image processing unit 103 determines the image processing based on imaging-protocol of the optical image set in the imaging preparation processing and stored in the storage 106 as the image processing to be performed on the optical image. In this example of explanation, based on the setting of the imaging-protocol for the left hand, the image processing unit 103 determines the image processing for rotating the optical image by the rotation angle adjusted in the imaging preparation processing and stored in association with the imaging-protocol.

In step S1304, the image processing unit 103 performs the image processing determined in step S1303 on the optical image obtained in step S1302. In this example of explanation, the image processing for rotating the optical image by the rotation angle adjusted in the imaging preparation processing is performed.

Since the processes of steps S1305 and S1306 are the same as the processes of steps S505 and S506 according to the first embodiment, the description thereof is omitted. When the obtaining unit 101 obtains the radiation image in step S1306, the process shifts to step S1307.

In step S1307, the obtaining unit 101 obtains the detected angle (yaw, pitch, and roll angles) of the radiation imaging apparatus 110 detected at the time of imaging the radiation image obtained in step S1306, from the angle detecting unit 111 of the radiation imaging apparatus 110.

In step S1308, the angle comparing unit 807 obtains the angle (set angle) of radiation imaging apparatus 110 obtained from the angle detecting unit 111 in step S1207 in the imaging preparation processing and stored in association with the imaging-protocol.

In step S1309, the angle comparing unit 807 compares the detected angle obtained in step S1307 with the set angle obtained in step S1308. More specifically, the angle comparing unit 807 compares the angle of the radiation imaging apparatus 110 at the time of imaging the radiation image obtained in the imaging processing with the angle of the radiation imaging apparatus 110 when the angle adjustment is completed in the imaging preparation processing.

In step S1310, the image processing unit 103 determines image processing to be performed for the radiation image based on the imaging-protocol selected in step S1300 and the angle comparison result obtained in step S1309. Specifically, the image processing unit 103 adjusts, according to the angle comparison result, the image processing based on imaging-protocol of the radiation image set in the imaging preparation processing and stored in storage 106, and determines the image processing to be performed on the radiation image. In this example of explanation, the image processing unit 103 adjusts the rotation angle adjusted based on the setting of the imaging-protocol for the left hand in the imaging preparation processing and stored in association with the imaging-protocol, according to the angle comparison result obtained in step S1309. Subsequently, the image processing unit 103 determines the image processing to rotate the radiation image by the rotation angle adjusted according to the angle comparison result as the image processing to be performed for radiation image.

The rotation angle adjustment processing related to the image processing according to the third embodiment will be described. First, the yaw angle in a case where the roll axis is assumed as the x-axis, the pitch axis is assumed as the y-axis, and the yaw axis is assumed as the z-axis in the recumbent imaging is described as an example. Here, it is assumed that the z-axis is positive in the downward direction, and the clockwise direction of the yaw angle is positive. In addition, it is assumed that the angle of the radiation imaging apparatus 110 stored in the storage 106 is 0 degrees, which is the reference point, and the yaw angle of the radiation imaging apparatus 110 detected by the angle detecting unit 111 when the radiation image is obtained in the imaging processing is +90 degrees. In this case, the radiation imaging apparatus 110 when the radiation image is obtained is rotated by 90 degrees clockwise from the state of the imaging preparation processing (reference point).

In this case, in step S1309, the angle comparing unit 807 obtains information indicating that the yaw angle is rotated by +90 degrees as the comparison result. Therefore, in step S1310, in order to compensate for the change from the reference point of the radiation imaging apparatus 110, the image processing unit 103 adjusts the yaw angle of the rotation angle set in the imaging preparation processing so that it is further rotated by −90 degrees. Subsequently, the image processing unit 103 determines the image processing for rotating the radiation image by the adjusted rotation angle as the image processing for radiation image. Here, the yaw angle is described as an example, but any of the yaw angle, roll angle, and pitch angle may be used depending on the imaging method such as standing imaging or lying imaging.

If the angle comparison result in step S1309 shows no change in the angle, the image processing unit 103 may determine the image processing for rotating the radiation image by the rotation angle adjusted in the imaging preparation processing and stored in association with imaging-protocol.

In step S1311, the image processing unit 103 performs the image processing determined in step S1310 on the radiation image obtained in step S1306. In this example of explanation, the image processing for rotating the radiation image by the rotation angle determined and adjusted in step S1310 is performed.

In step S1312, the display controlling unit 105 causes the display unit 150 to display the radiation image subjected to the image processing. When the processing in step S1312 is completed, the controlling apparatus 800 ends the imaging processing.

In the third embodiment, the image processing unit 103 determines the image processing of the radiation image in step S1310 based on the angle comparison result by the angle comparing unit 807, and performs the determined image processing on the radiation image in step S1311. On the other hand, in step S1310, the image processing for rotating the radiation image by the rotation angle stored in association with imaging-protocol may be determined. In this case, the image processing unit 103 may perform further rotation processing based on the angle comparison result on a radiation image rotated by the rotation angle stored in association with imaging-protocol, in step S1311.

As described above, the obtaining unit 101 according to the third embodiment obtains a detection angle which is an example of orientation information indicating the orientation of the radiation imaging apparatus 110 that images the radiation image. The image processing unit 103 adjusts the image processing for the radiation image based on the orientation information. More specifically, the image processing unit 103 adjusts the image processing for the radiation image according to the result of comparison between the obtained orientation information and the set angle which is an example of information indicating the orientation of radiation imaging apparatus 110 regarding the image processing for the radiation image.

With this configuration, the controlling apparatus 800 according to the third embodiment can adjust the image processing even if the physical angle of the radiation imaging apparatus 110 at the time of imaging the radiation image is different from the angle of the radiation imaging apparatus 110 stored at the time of setting the image processing. Therefore, the controlling apparatus 800 can compensate for the difference in the angle of the radiation imaging apparatus 110 and provide the optical image and the radiation image in a manner in which the optical image and the radiation image correspond to each other.

### Fourth Embodiment

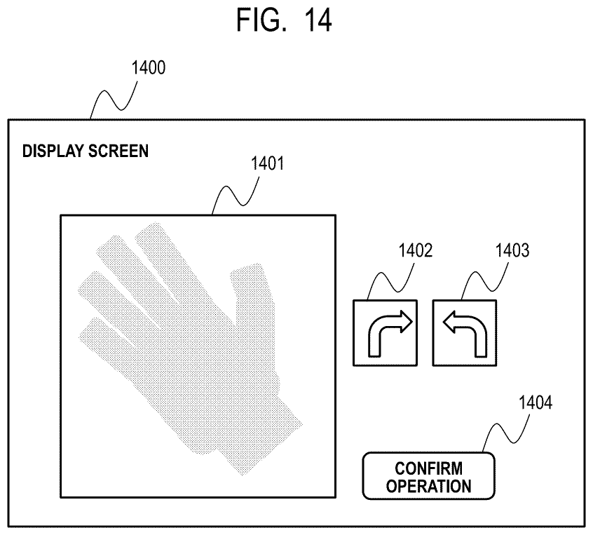

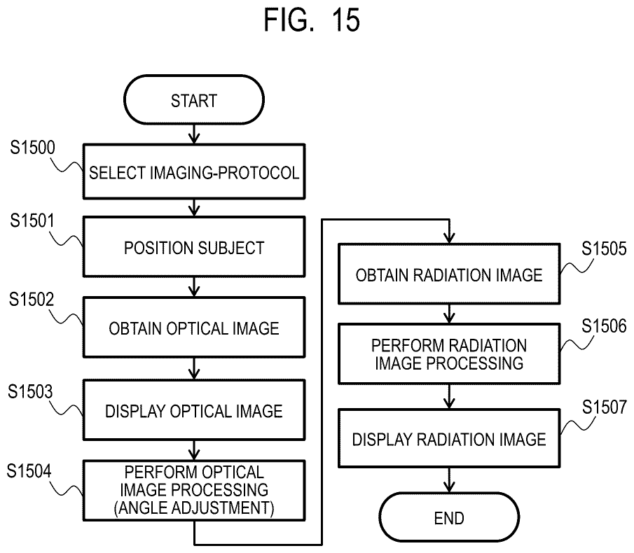

In the first embodiment, the image processing to be performed on the optical image and the radiation image at the time of imaging is performed according to a preset content. On the other hand, in a fourth embodiment of the present disclosure, an example in which operation information of the image operation manually performed by the user at the time of imaging is obtained and image processing based on the obtained operation information is performed will be described. A radiation imaging system, an image processing apparatus, and an image processing method according to the fourth embodiment will be described below with reference to FIG. 14 and FIG. 15. Description of the same configuration, functions, and operations which are same as those of the first embodiment will be omitted, and differences between the fourth embodiment and the first embodiment will be mainly described.

### <Configuration of Radiation Imaging System>

The configuration of the radiation imaging system according to the fourth embodiment is the same as that of the radiation imaging system according to the first embodiment. However, the obtaining unit 101 can obtain operation information from the user input via the operation unit 160 regarding image processing of the optical image and the radiation image. The image processing unit 103 can perform any image processing on the optical image and the radiation image based on the operation information obtained by obtaining unit 101.

### <Imaging Processing>

The imaging processing according to the fourth embodiment will be described with reference to FIG. 14 and FIG. 15. In the fourth embodiment, since the operation information related to the image processing is obtained at the time of imaging, the imaging preparation processing performed in the first embodiment is not performed. FIG. 14 is a diagram for illustrating an example of the imaging display screen according to the fourth embodiment. FIG. 15 is a flowchart showing an example of the procedure of imaging processing according to the fourth embodiment.

Since the processes of steps S1500 to S1502 according to the fourth embodiment are the same as the processes of steps S500 to S502 according to the first embodiment, description thereof is omitted. If the obtaining unit 101 obtains the optical image in step S1502, the process shifts to step S1503. Here, for convenience of explanation, an example in which imaging-protocol for the left hand is selected will be described.

In step S1503, the display controlling unit 105 causes the optical image obtained by the obtaining unit 101 to be displayed on a display screen 1400 of the display unit 150. At this time, the display controlling unit 105 causes the obtained optical image to be displayed on an image display area 1401 of the display screen 1400.

In step S1504, the obtaining unit 101 obtains operation information for adjusting the rotation angle of the optical image when the user presses a right rotation button 1402 or a left rotation button 1403 so that the desired image display is achieved while checking the optical image displayed on the image display area 1401. Further, the obtaining unit 101 obtains operation information of the rotation angle of the optical image when the user presses an operation confirmation button 1404 after the display of the optical image becomes the desired image display, as final operation information. The obtaining unit 101 causes the storage 106 to store the adjusted rotation angle corresponding to the final operation information.

In step S1505, the obtaining unit 101 obtains the radiation image as in step S506 according to the first embodiment. If the obtaining unit 101 obtains the radiation image in step S1505, the process shifts to step S1506.

In step S1506, the image processing unit 103 performs image processing for rotating the radiation image so that the optical image and the radiation image correspond to each other based on the rotation angle stored in the storage 106 in step S1504. Here, the image processing unit 103 may perform the image processing on the radiation image, which is similar to the image processing performed on the optical image in step S1504. If the deviation of the appearances of the optical image and the radiation image obtained by the radiation system, for example, the angular deviation of the imaging ranges of each other, is known, the image processing unit 103 may adjust the rotation angle so as to compensate for the deviation when performing the rotation processing of radiation image, and perform the rotation processing.

In step S1507, the display controlling unit 105 causes the display unit 150 to display the radiation image on which the image processing is performed. The controlling apparatus 100 ends the imaging processing when the process in step S1507 is completed.

In the fourth embodiment, the image processing unit 103 performs rotation processing of the radiation image based on a rotation angle corresponding to operation information. However, the image processing according to the fourth embodiment is not limited to this. For example, in step S1504, the image processing unit 103 may recognize the orientation of the subject in the optical image from the optical image of which the rotation angle has been adjusted, by the same processing as in the first embodiment, and store the orientation of the subject in the storage 106. In this case, in step S1506, the image processing unit 103 may recognize the orientation of the subject in the radiation image by the same processing as in the first embodiment. Subsequently, image processing unit 103 may perform rotation processing on the radiation image so that the subject in the radiation image has the orientation of the subject stored in the storage 106 in step S1504.

In the fourth embodiment, the image processing based on the image processing for optical image is applied to the radiation image. On the other hand, image processing unit 103 may apply the image processing based on the content of the image processing for radiation image to the optical image. In this case, the image processing unit 103 may obtain operation information when the user adjusts the rotation angle of the radiation image while checking the radiation image, and apply the rotation processing based on the rotation angle corresponding to the operation information to the optical image. In addition, as described above, the image processing unit 103 may recognize the orientation of the subject from the radiation image of which the rotation angle is adjusted, and perform the rotation processing of the optical image so that the subject in the optical image has the recognized orientation.

As described above, the obtaining unit 101 according to the fourth embodiment obtains operation information related to one of the image processing of the optical image or the image processing of the radiation image. Further, the image processing unit 103 determines the one of the image processing of the optical image and the image processing of the radiation image based on the operation information. Furthermore, the image processing unit 103 determines the other of the image processing of the optical image and the image processing of the radiation image based on the one of the image processing of the optical image and the image processing of the radiation image determined based on the operation information. Even with this configuration, the controlling apparatus 100 can make the appearances of the optical image and the radiation image correspond to each other. Therefore, the controlling apparatus 100 can reduce the burden on the patient and the user by providing an image that is easy for the user to check and reducing the need to have the patient move or the need to adjust the image.

In the fourth embodiment, the obtaining unit 101 obtains the operation information from the user input via the operation unit 160 relating to the image processing of the optical image and the radiation image. On the other hand, an obtaining unit for obtaining operation information from the user relating to image processing of the optical image and an obtaining unit for obtaining operation information from the user relating to image processing of the radiation image may be separately provided. In this case, the obtaining unit 101 may be provided as a component that includes these obtaining units.

Similarly, in the fourth embodiment, the image processing unit 103 performs the image processing on the optical image and the radiation image based on the operation information obtained by the obtaining unit 101. On the other hand, an image processing unit for performing image processing on the optical image based on the operation information related to the image processing of the optical image and an image processing unit performing image processing on the radiation image based on the operation information related to the image processing of the radiation image may be separately provided. In this case, the image processing unit 103 may be provided as a component that includes these image processing units.

### Modification

The rotation angle adjusted in step S1504 can be stored in the storage 106 as information indicating the image processing in association with the imaging-protocol selected in step S1500. In this case, when the image processing unit 103 performs the imaging processing according to the imaging-protocol to which the information indicating the image processing is associated, the image processing may be applied to an optical image and a radiation image obtained in subsequent imaging processing based on the information indicating image processing stored in the storage 106. In this case, before applying the image processing, the display controlling unit 105 may cause the display unit 150 to display a warning such as a dialog box to inform that the image processing performed previously is to be applied. As described above, the orientation of the subject in the image of which the rotation angle is adjusted may be stored in the storage 106 as the information indicating the image processing in association with the imaging-protocol selected in step S1500.

As described above, the controlling apparatus 100 according to this modification further includes a storage 106 that stores information indicating the determined image processing of the optical image and the image processing of the radiation image. Further, based on the stored information, the image processing unit 103 performs the image processing on an optical image and a radiation image, which are obtained later than the obtained optical image and the radiation image. In such a case, the image processing adjusted once can be applied to subsequent image processing, thereby improving the convenience of the controlling apparatus 100.

The setting screens and the display screens described in the embodiments 1 to 4 are examples. Therefore, the arrangement and the display mode of various images, buttons, etc. on the setting screens and the display screens displayed on the display unit 150 may set freely. For example, the display controlling unit 105 may cause the optical image and the radiation image to be displayed on the display unit 150. In this case, the controlling apparatus 100 can more efficiently support the user to confirm both optical image and radiation image and to judge whether or not the position alignment of the subject is appropriate.

According to the embodiments 1 to 4, the image processing can be performed on at least one of an optical image and a radiation image so that the optical image and the radiation image correspond to each other.

In the above-mentioned learned model for recognizing the orientation of the subject, learned model for recognizing the imaged site, and the like, it is conceivable for the magnitude of intensity values, and the order and slope, positions, distribution, and continuity of bright sections and dark sections and the like of an image that is input data to be extracted as a part of feature values and used it for inference processing.

Moreover, the above-mentioned learned model (inferrer) for recognizing the orientation of the subject, learned model for recognizing the imaged site, and the like can be provided in the controlling apparatus 100. The learned models may be, for example, constituted by a software module that is executed by a processor such as a CPU, an MPU, a GPU, or an FPGA, or may be constituted by a circuit that serves a specific function such as an ASIC. Further, the learned models may be provided in another apparatus such as a server connected to the controlling apparatus 100. In this case, the controlling apparatus 100 can use the learned model by connecting to the server or the like that includes the learned models through any network such as the Internet. The server that includes the learned models may be, for example, a cloud server, a FOG server, an edge server, or the like. No that, in a case where a network in a facility, or within premises in which the facility is included, or within an area in which a plurality of facilities are included or the like is configured to enable wireless communication, for example, the reliability of the network may be improved by configuring the network to use radio waves in a dedicated wavelength band allocated to only the facility, the premises, or the area or the like. Further, the network may be constituted by wireless communication that is capable of high speed, large capacity, low delay, and many simultaneous connections.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

The processor or circuit may include a central processing unit (CPU), a micro processing unit (MPU), a graphics processing unit (GPU), an application specific integrated circuit (ASIC), or a field programmable gateway (FPGA). The processor or circuit may also include a digital signal processor (DSP), a data flow processor (DFP), or a neural processing unit (NPU).

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

1. An image processing apparatus comprising:
an obtaining unit configured to obtain an optical image which is obtained by optically imaging a subject and a radiation image which is obtained by imaging the subject with radiation; and
an image processing unit configured to perform image processing including at least one of rotation processing, extraction processing and scaling processing on the optical image so that the optical image and the radiation image correspond to each other.

2. The image processing apparatus according to claim 1, further comprising:
a selecting unit configured to select an imaging-protocol relating to imaging of the subject,
wherein the image processing unit is configured to perform the image processing according to the imaging-protocol on the optical image and the radiation image so that the optical image and the radiation image correspond to each other.

3. The image processing apparatus according to claim 2, further comprising:
a recognizing unit configured to recognize an imaged site of the subject using the optical image; and
a judging unit configured to judge whether the recognized imaged site matches with site information indicating an imaged site of the subject associated with the imaging-protocol,
wherein in a case where it is judged that the recognized imaged site does not match with the site information, the image processing unit does not perform the image processing on the optical image.

4. The image processing apparatus according to claim 3, wherein in the case where it is judged that the recognized imaged site does not match with the site information, the obtaining unit does not obtain the radiation image.

5. The image processing apparatus according to claim 3, further comprising:
a display controlling unit configured to cause a warning to be displayed on a display unit in the case where it is judged that the recognized imaged site does not match with the site information.

6. The image processing apparatus according to claim 3, wherein the recognizing unit is configured to recognize the imaged site of the subject by using an optical image having an imaging range wider than an imaging range of the optical image on which the image processing is performed.

7. The image processing apparatus according to claim 3, wherein the recognizing unit is configured to recognize the imaged site of the subject in the obtained optical image by using an output from a learned model obtained by inputting the obtained optical image into the learned model which is obtained by using training data including an optical image and information indicating an imaged site of a subject in an optical image.

8. The image processing apparatus according to claim 1, wherein the image processing unit is configured to:
recognize an orientation of the subject in the optical image and perform the image processing on the optical image so that the subject in the optical image has a predetermined orientation; and
recognize an orientation of the subject in the radiation image and perform the image processing on the radiation image so that the subject in the radiation image has a predetermined orientation.

9. The image processing apparatus according to claim 8, wherein, in a case where the subject is a human hand, the image processing unit is configured to:
estimate positions of a fingertip and a wrist of the subject in the optical image and the radiation image;
recognize an orientation of the subject in the optical image based on the positions of the fingertip and wrist of the subject in the optical image; and
recognize an orientation of the subject in the radiation image based on the positions of the fingertip and the wrist of the subject in the radiation image.

10. The image processing apparatus according claim 9, wherein, in the case where the subject is a human hand, the image processing unit is configured to:
further estimate position of a base of a finger of the subject in the optical image and the radiation image;
obtain a rotation axis of the optical image based on the positions of the fingertip, the base of the finger, and the wrist of the subject in the optical image and perform the rotation processing on the optical image about the rotation axis of the optical image; and
obtain a rotation axis of the radiation image based on the positions of the fingertips, the base of fingers, and the wrist of the subject in the radiation image and perform the rotation processing on the radiation image about the rotation axis of the radiation image.

11. The image processing apparatus according to claim 8, wherein image processing unit is configured to:
recognize the orientation of the subject in the obtained optical image by using an output from a learned model obtained by inputting the obtained optical image into the learned model which is obtained by using training data including an optical image and information indicating an orientation of a subject in an optical image; and
recognize the orientation of subject in the obtained radiation image by using an output from a learned model obtained by inputting the obtained radiation image into the learned model which is obtained by using training data including a radiation image and information indicating an orientation of a subject in a radiation image.

12. The image processing apparatus according to claim 1, wherein:
the obtaining unit is configured to obtain operation information relating to one of image processing of the optical image and image processing of the radiation image; and
the image processing unit is configured to determine the one of the image processing of the optical image and the image processing of the radiation image based on the operational information.

13. The image processing apparatus according to claim 12, wherein the image processing unit is configured to determine the other of the image processing of the optical image and the image processing of the radiation image based on the one of the image processing of the optical image and the image processing of the radiation image determined based on the operation information.

14. The image processing apparatus according to claim 13, further comprising:
a storage arranged to store information indicating the determined image processing of the optical image and the determined image processing of the radiation image,
wherein the image processing unit is configured to perform the image processing on an optical image and a radiation image obtained later than the obtained optical image and the obtained radiation image based on the stored information.

15. The image processing apparatus according to claim 1, wherein:
the obtaining unit is configured to obtain orientation information indicating an orientation of a radiation imaging apparatus arranged to image the radiation image; and
the image processing unit is configured to adjust the image processing for the radiation image based on the orientation information.

16. The image processing apparatus according to claim 15, wherein the image processing unit is configured to adjust the image processing for the radiation image according to a comparison result between the obtained orientation information and information indicating an orientation of the radiation imaging apparatus relating to the image processing for the radiation image.

17. The image processing apparatus according to claim 1, wherein the image processing unit is configured to perform the image processing on the optical image so that one of an orientation of the subject in the optical image and an orientation of the subject in the radiation image becomes the other.

18. A radiation imaging system comprising:
an optical imaging apparatus arranged to optically image a subject;
a radiation imaging apparatus arranged to image the subject with radiation; and
the image processing apparatus according to claim 1.

19. An image processing method comprising:
obtaining an optical image which is obtained by optically imaging a subject and a radiation image which is obtained by imaging the subject with radiation; and
performing image processing including at least one of rotation processing, extraction processing, and scaling processing on the optical image so that the optical image and the radiation image correspond to each other.

20. A non-transitory computer-readable storage medium having stored thereon a program for causing, when performed by a computer, the computer to perform respective steps of the image processing method according to claim 19.
